(19) United States
(12) Patent Application Publication
(10) Pub. No.: US 2011/0245680 A1
(43) Pub. Date: Oct. 6, 2011

(54) ULTRASOUND APPARATUS WITH HUMIDITY PROTECTION

(75) Inventor: Thomas Hunt, Pelham, NH (US)

(73) Assignee: KONINKLIJKE PHILIPS ELECTRONICS N.V., EINDHOVEN (NL)

(21) Appl. No.: 13/132,627

(22) PCT Filed: Dec. 10, 2009
(86) PCT No.: PCT/IB2009/055657

§ 371 Date: Jun. 3, 2011

Related U.S. Application Data
(60) Provisional application No. 61/122,602, filed on Dec. 15, 2008.

(51) Int. Cl. A61B 8/00

(52) U.S. Cl. 600/459

(57) ABSTRACT

An ultrasound apparatus, a medical device comprising such ultrasound apparatus and a method for operating an ultrasound apparatus are proposed. The ultrasound apparatus (10) comprises a power supply (1), system electronics (2) for controlling e.g. at least one of an ultrasound generator and an ultrasound detector, a humidity sensor (5) and a controller (3). The controller (3) is adapted to connect the power supply (1) to the system electronics (2) only in case the humidity sensor (5) detects a humidity below a predetermined threshold. By implementation of such humidity control within the ultrasound apparatus, it can be prevented to operate the ultrasound apparatus under humid conditions which, due to water condensation, may be dangerous for damaging the system electronics (2) of the ultrasound apparatus (10).

### FIELD OF THE INVENTION

The present invention relates to an ultrasound apparatus, a medical device comprising such ultrasound apparatus and to a method of operating an ultrasound apparatus.

### BACKGROUND OF THE INVENTION

Ultrasound apparatus are frequently used in medical applications in order to investigate a patient's tissue. As, on the one side, such ultrasound apparatus may be used for various medical applications and as, on the other side, such ultrasound apparatus may be comparatively expensive, there have been approaches to provide compact ultrasound systems which may be easily transported from one location to another and which may even have a portable power supply such as a battery. Such compact ultrasound systems may be easily transferred between different application locations and may operate under different environmental conditions.

In a user manual of such compact ultrasound systems, the following statement may be found: “If systems, transducers and peripherals have been in an environment of 10° C. or below, allow them to reach room temperature before connecting or turning them on. The manufacturer recommends allowing 24 hours for complete normalization.”

However, one advantage of a compact ultrasound system should be the flexibility of their application and the ease with which it can be moved for example from building to building. Putting a 24 hour “normalization” time on that may greatly reduce that advantage.

### SUMMARY OF THE INVENTION

Accordingly, there may be a need for an improved ultrasound apparatus, an improved medical device comprising such ultrasound apparatus and an improved method of operating an ultrasound apparatus. Particularly, there may be a need for an improved ultrasound apparatus which allows safe operation under various environmental conditions differing in temperature and/or humidity. Furthermore, there may be a need for an improved ultrasound apparatus which may reduce the time a user has to wait until the apparatus may be safely operated after transferring it from one environment to another environment.

These needs may be met by the subject-matter according to the independent claims. Advantageous embodiments of the present invention are described in the dependent claims.

According to a first aspect of the present invention, an ultrasound apparatus comprising a power supply, system electronics for controlling e.g. at least one of an ultrasound generator and an ultrasound detector, a humidity sensor and a controller is provided. Therein, the controller is adapted to connect the power supply to the system electronics only in case the humidity sensor detects a humidity below a predetermined threshold.

A gist of the present invention may be seen as being based on the following idea:

It has been observed that condensation on the electronics of an ultrasound apparatus may cause damage if the system is powered up. Furthermore, a user usually has no way of knowing when it is safe to power up an ultrasound apparatus after transferring it from one environment to another environment, for example after bringing the apparatus inside an air conditioned environment from a humid outside environment. In prior art ultrasound systems, it was therefore recommended to wait 24 hours to be certain before powering up the system.

It is therefore proposed herein to integrate a humidity sensor into an ultrasound apparatus wherein the humidity sensor may detect a degree of humidity or condensation which might be dangerous for the system electronics of the ultrasound apparatus. A controller is adapted such that the system electronics are provided with electricity from the power supply only if a level of humidity detected by the humidity sensor is below a predetermined threshold. Otherwise, if a detected humidity level is above the threshold, the controller may prevent the booting or energizing of the system electronics. In such case, the control may repeatedly check the humidity level and may enable an electrical connection between the power supply and the system electronics as soon as the humidity has decreased below the predetermined threshold.

Accordingly, the provision of the humidity sensor and the corresponding adaption of the controller may, on the one side, ensure a secure operation of the ultrasound apparatus even after environment changes and increased humidity levels and, on the other side, may minimize the time a user may have to wait for complete normalization before being able to securely use the ultrasound apparatus.

In the following, possible features and advantages of embodiments of the proposed ultrasound apparatus will be described.

The power supply of the ultrasound apparatus may be any kind of power supply being able to energize the system electronics and possibly also other electrical components of the ultrasound apparatus such as an ultrasound generator or an ultrasound detector. For example, the power supply may be a mains supply circuit or an AC/DC converter having a plug which can be connected to an electricity network. Alternatively, the power supply can be provided as a battery or accumulator which may be integrated into or attached to the ultrasound apparatus.

The system electronics may be adapted for controlling at least one of an ultrasound generator and an ultrasound detector. Furthermore, the system electronics may be adapted for controlling further components of the ultrasound apparatus such as for example additional computing units, displaying units, etc. The system electronics may be adapted for both providing electrical energy to such ultrasound apparatus components as well as for providing electrical control signals for controlling the operation of such components. The system electronics may be implemented in conventional printed circuit boards and may be at least in parts highly integrated.

The humidity sensor may be any kind of sensor which is able to detect a humidity or condensation level within a specific space or environment. There are various methods of measuring humidity, which may be expressed as the proportion of water vapour in air, quoted as a percentage. The electrical properties of an absorbent material may change with humidity, and the variation in conductivity or capacitance, can be measured. The accuracy of the measurement device should be considered when setting the maximum allowable operational humidity specification for the ultrasound apparatus, for example: if the maximum allowable operating humidity level of the ultrasound apparatus is 80% RH and the chosen humidity sensor has an accuracy of +/−5% RH, then the maximum allowable measured humidity should be less than 75% RH in order to allow the ultrasound apparatus to turn on.

The controller of the ultrasound apparatus shall be adapted to enable or interrupt an electrical connection between the power supply and the system electronics depending on a humidity level detected by the humidity sensor. A predetermined threshold value may be set or stored for the controller. The controller may then compare the actual value of the humidity detected by the humidity sensor with the predetermined threshold value. In case, the actually measured value is lower than the threshold, the controller may enable an electrical connection between the power supply and the system electronics. After such energizing of the system electronics, the ultrasound apparatus may boot and may then be used for the desired ultrasound application.

The controller may repeatedly, preferably periodically, check the humidity, i.e. receive measurement values from the humidity sensor, and may each time determine whether the connection between the power supply and the system electronics may be established or not. Depending on the current status of the system, the controller can then maintain or change the connection/disconnection between the power supply and the system electronics.

According to an embodiment of the present invention, the ultrasound apparatus further comprises a main switch to be operated by a user for turning ON the ultrasound apparatus. Therein, the controller may be adapted to, upon operation of the main switch, check a detection signal from the humidity sensor first and to connect the power supply to the system electronics only in case the humidity sensor detects a humidity below a predetermined threshold.

In other words, when the ultrasound apparatus is originally turned OFF, a user may operate a main switch thereby indicating that the ultrasound apparatus is to be turned ON. However, before actually energizing the system electronics, the controller will ask the humidity sensor for the actual current humidity value and, based on the obtained humidity value, may determine whether the system electronics may be safely operated or whether the humidity value is too high such that the risk of condensation of water comprised in the air may prevent such secure operation.

In case, the controller primarily prevents the power supply to the system electronics due to a detected humidity value being above the predetermined threshold, the controller may then repeatedly acquire updated humidity measurement values from the humidity sensor and check whether the humidity has decreased in the meantime. As soon as the humidity is below the predetermined threshold, the system electronics may then be energized and the ultrasound apparatus may boot and then be operated.

According to a further embodiment of the present invention, the humidity sensor is adapted to detect at least one of a humidity and a condensation level in a close proximity to the system electronics. The humidity sensor may be installed for example in direct proximity to the system electronics, for example in a distance of a few cm, e.g. 10 cm, or less, or may be even implemented on the same printed circuit board as used for the system electronics. In the usual case that the system electronics are provided within a closed housing optionally enclosing also other components of the ultrasound apparatus, the humidity sensor can be arranged within such housing in order to measure the humidity of air comprised in the housing.

According to a further embodiment of the present invention, the ultrasound apparatus further comprises a drying device for drying the system electronics. Therein, the controller is adapted to operate the drying device in case the humidity sensor detects a humidity above the predetermined threshold.

For example, the drying device may comprise a fan supplying air to the system electronics. Such air may be drawn from an environment outside the housing enclosing the system electronics which environmental air may be drier than the air originally comprised within the housing. Accordingly, by supplying such dry air, the environment of the system electronics may be dried down to a humidity level below the predetermined threshold for secure operation. Alternatively or additionally, the drying device may comprise a heater. Such heater may heat directly the electronics to prevent or remove water condensation thereof. Alternatively, the heater may heat air supplied from an outside environment for example by an additional fan.

According to a further embodiment of the present invention, the ultrasound apparatus further comprises an indication device for providing an indication to a user. Therein the controller is adapted to operate the indication device depending on a detection signal from the humidity sensor. For example, the controller may operate the indication device in case the humidity sensor detects a humidity above the predetermined threshold.

The indication device may be any device which may emit a signal that can be recognized by a user. For example, such signal can be a visual or auditable signal. For example, the indicating device can be a light emitting device such as an LED or a sound emitting device such as a loudspeaker. While the controller will not enable the energizing of the system electronics upon the detection of an excessive humidity level, the controller may instead operate the indication device in order to indicate to a user, who has for example previously operated the main switch, that the ultrasound apparatus cannot be started at the moment due to the excessive humidity level but that the humidity level will be continuously monitored and that the ultrasound apparatus will be started as soon as the humidity level has sufficiently decreased.

According to a further embodiment of the present invention, the controller of the ultrasound apparatus is adapted to operate safely in an environment having a humidity level above the predetermined threshold. In other words, the controller may be designed or protected in such a way that it can operate under humid environmental conditions which would be possibly dangerous for the system electronics. While it may be complicated or impossible and expensive to protect the entire system electronics against humidity influences, it may be easily possible to protect the controller such that no excessive humidity can reach the controller or to design the controller in such a robust way that increased humidity levels may not negatively influence its operation.

According to a further embodiment of the present invention, an electronic circuit of the control is protected against humidity by a conformal coating. Such conformal coating may seal the controlled electronic circuit against humidity.

Conformal coatings may be materials applied in thin layers of typically a few micrometers or a fraction of a mm onto printed circuits or other electronic substrates. They may provide environmental and mechanical protection to e.g. significantly extend the life of the components and circuitry. Conformal coatings are traditionally applied by dipping, spraying or simple flow coating, and increasing by by select coating or robotic dispensing.

Conformal coatings may protect electronic printed circuit hoards from moisture and contaminants, preventing short circuits and corrosion of conductors and solder joints, They may also minimize dendritic growth and the electromigration of metal between conductors. In addition, the use of conformal coatings may protect circuits and components from abrasion and solvents. Stress relief may also be provided, as well as protection of the insulation resistance of the circuit board.

According to a further embodiment of the present invention, the power supply of the ultrasound apparatus is implemented as a battery. Such battery may be integrated into the ultrasound apparatus or may be attached thereto. The battery may be small enough such that the ultrasound apparatus may be easily transported. However, the battery may supply sufficient electrical energy for normal operation of the ultrasound apparatus. Accordingly, such battery may render the ultrasound apparatus independent of any electricity network. Preferably, the battery is rechargeable.

According to a further aspect of the present invention, a medical device comprising the above-described ultrasound apparatus is proposed. Such medical device may be any device adapted for examination or treatment of patients using ultrasonic waves. For example, the medical device may be a compact and therefore portable echocardiography or cardiovascular ultrasound system.

According to a further aspect of the present invention, a method of operating an ultrasound apparatus is provided. Therein, the ultrasound apparatus comprises a power supply and system electronics for controlling at least one of an ultrasound generator and an ultrasound detector. Furthermore, it comprises a main switch for turning ON the ultrasound apparatus and a humidity sensor. The method comprises the step of, upon operating the main switch, checking a detection signal from the humidity sensor first and then connecting the power supply to the system electronics only in case the humidity sensor detects a humidity below a predetermined threshold.

Expressed in other words, features of the invention and its embodiments may be summarized as follows: The proposed ultrasound apparatus may automatically detect a condition of humidity/condensation that may be dangerous to the system electronics and may prevent the system from booting up until that condition has been corrected, i.e. until complete normalization is obtained. Furthermore, the proposed ultrasound apparatus may enable parts of the apparatus such as a fan or a heater, to turn ON to accelerate the normalization of the environment and finally, the proposed ultrasound apparatus may provide user feedback that a high humidity environment has prevented the system from booting for example with some form of simple visual indication like a blinking LED.

It has to be noted that features and advantages of the present invention have been described with respect to different embodiments of the invention. Particularly, some features are described with respect to the ultrasound apparatus while other features are described with respect to the method of operating an ultrasound apparatus. However, a person skilled in the art will gather from the above and the following description that, unless other notified, in addition to any combination or features belonging to one embodiment also any combinations between features relating to different embodiments or to a manufacturing method is considered to be disclosed with this application.

### BRIEF DESCRIPTION OF THE DRAWING

Features and advantages of the present invention will be described with respect to a specific embodiment as shown in the accompanying drawing but to which the invention shall not be limited.

FIG. 1 shows a schematic representation of an ultrasonic apparatus according to an embodiment of the present invention.

### DETAILED DESCRIPTION OF THE PREFERRED EMBODIMENT

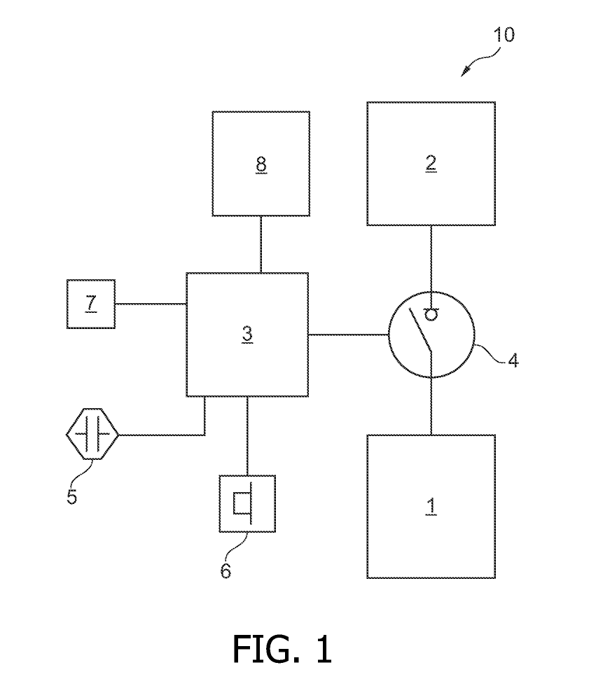

The exemplary ultrasound apparatus 10 shown in FIG. 1 comprises a Power Supply 1, which could include a battery, system electronics 2 and a controller 3. The controller 3 controls a Switch 4 which connects the Power Supply 1 to the System Electronics 2. The controller 3 is also connected to a humidity sensor 5. The humidity sensor 5 is comprised in a housing (not shown) enclosing the system electronics 2 and possibly other components of the ultrasound apparatus 10.

The ultrasound apparatus 10 further comprises a drying device 8 in the form of a fan for supplying dry air to the system electronics 2. Furthermore, the ultrasound apparatus 10 comprises an indicating device 7 in the form of an LED. This indicating device 7 is connected to and controlled by the controller 3.

Finally, the ultrasound apparatus 10 comprises a main switch 6 serving as an ON/OFF button 6 which is connected to the controller 3.

The battery-operated compact ultrasound apparatus 10 is usually never completely turned OFF. There is a stand-by circuit that monitors the ON/OFF main switch 6 and orchestrates the power-ON sequence. As such stand-by circuit included in the controller 3 includes the ability to monitor the humidity/condensation level inside the housing of the ultrasound apparatus 10, it is able to determine when it is safe to turn ON the apparatus by energizing the system electronics 2 without risk of condensation damage. Additionally, the controller 3 can initiate steps to reduce in-system humidity levels prior to turning ON the rest of the circuitry. For example, a fan and/or a heater included in the drying device 8 can be operated. Finally, the controller 3 enables some user feedback that the system is in a drying-out cycle by blinking those LED of the indicating device 7 so that the user would know why the system does not turn ON.

In case the user tries to turn ON the apparatus by operating the main switch 6, the controller 3 checks the humidity/condensation sensor 5 in order to see that the conditions are OK for turning ON. If they are not, the controller 3 enables the drying device 8 and starts to blink the LED indicating that there is access humidity present. The controller 3 then continues to pull the humidity/condensation sensor until the environment in the electronics have completely normalized and then proceeds to enable the power supply 1 to the rest of the system electronics 2.

The proposed ultrasound apparatus 10 may be useful for medical devices that are frequently moved between different facilities and likely to encounter different environmental conditions.

Finally, it should be noted that the terms “comprising”, “including”, etc. do not exclude other elements or steps and the terms “a” or “an” do not exclude a plurality of elements. Also, elements described in association with different embodiments may be combined. It should also be noted that reference signs in the claims should not be construed as limiting the scope of the claims.

1. An ultrasound apparatus (10) comprising:
a power supply (1);
system electronics (2);
a humidity sensor (5);
a controller (3);
wherein the controller (3) is adapted to connect the power supply (1) to the system electronics (2) only in case the humidity sensor (5) detects a humidity below a predetermined threshold.

2. The ultrasound apparatus (10) of claim 1, further comprising a main switch (6) to be operated by a user for turning ON the ultrasound apparatus (10);
wherein the controller (3) is adapted to, upon operation of the main switch (6), check a detection signal from the humidity sensor (5) first and to connect the power supply (1) to the system electronics (2) only in case the humidity sensor detects (5) a humidity below a predetermined threshold.

3. The ultrasound apparatus (10) of claim 1, wherein the humidity sensor (5) is adapted to detect at least one of a humidity and a condensation level in a close proximity to the system electronics (2).

4. The ultrasound apparatus (10) of claim 1, further comprising a drying device (8) for drying the system electronics (2);
wherein the controller (3) is adapted to operate the drying device (8) in case the humidity sensor (5) detects a humidity above the predetermined threshold.

5. The ultrasound apparatus (10) of claim 1, further comprising an indication device (7) for providing an indication to a user;
wherein the controller (3) is adapted to operate the indication device (7) depending on a detection signal from the humidity sensor (5).

6. The ultrasound apparatus (10) of claim 1, wherein the controller (3) is adapted to operate safely in an environment having a humidity level above the predetermined threshold.

7. The ultrasound apparatus (10) of claim 1, wherein an electronic circuit of the controller (3) is protected against humidity by a conformal coating.

8. The ultrasound apparatus (10) of claim 1, wherein the power supply (1) is implemented with a battery

9. A medical device comprising an ultrasound apparatus (10) according to claim 1.

10. A method of operating an ultrasound apparatus (10), wherein the ultrasound apparatus (10) comprises:
a power supply (1);
system electronics (2);
a humidity sensor (5);
a main switch (6) for turning ON the ultrasound apparatus (10);
wherein the method comprises:
upon operation of the main switch (6), checking a detection signal from the humidity sensor (5) first and connecting the power supply (1) to the system electronics (2) only in case the humidity sensor (5) detects a humidity below a predetermined threshold.
